(19) United States
(12) Patent Application Publication
(10) Pub. No.: US 2005/0049794 A1
(43) Pub. Date: Mar. 3, 2005

(54) Processes for producing optimized pharmacophores

(76) Inventors: John H. van Drie, Andover, MA (US); Jeffrey Weilee Peng, Granger, IN (US)

Correspondence Address: Nandakumar Govindaswamy;Vertex Pharmaceuticals Incorporated, 130 Waverly Street, Cambridge, MA, 02139, US

(21) Appl. No.: 10/838,705

(22) Filed: Apr. 29, 2004

Related U.S. Application Data
(63) Continuation of application No. PCT/US02/34512, filed on Oct. 29, 2002.
(60) Provisional application No. 60/350,080, filed on Oct. 29, 2001.

(51) Int. Cl. C12Q 1/68; G06F 19/00; G01N 33/48; G01N 33/50; G01N 31/00

(52) U.S. Cl. 702/19; 702/22

(57) ABSTRACT

The present invention relates to processes for producing an optimized pharmacophore for a target protein. The present invention also relates to processes for identifying compounds having an affinity to a target protein. The present invention also relates to processes for designing a ligand for a target protein using the optimized pharmacophore of the present invention. The present invention also provides a computer for use in designing a ligand for a target protein using the optimized pharmacophore of the present invention.

### CROSS-REFERENCE TO RELATED APPLICATIONS

The present application is a continuation, and claims the benefit, of International PCT Application No. PCT/US02/34512, filed Oct. 29, 2002, which claims the benefit of U.S. provisional application No. 60/350,080, filed Oct. 29, 2001, the entire disclosure of these two documents being incorporated herein by reference.

### TECHNICAL FIELD OF THE INVENTION

The present invention relates to processes for producing an optimized pharmacophore for a target protein. The present invention also relates to processes for identifying compounds having an affinity to a target protein. The present invention also relates to processes for designing a ligand for a target protein using the optimized pharmacophore of the present invention. The present invention also provides a computer for use in desgining a ligand for a target protein using the optimized pharmacophore of the present invention.

### BACKGROUND OF THE INVENTION

Structure-based drug design (“SBDD”) has transformed the process of drug discovery. Through iterations of determination of the high-resolution X-ray structures of drug targets with small molecules, chemists have been able to accelerate the process of designing molecules with the desired biological properties. The rapid discovery of therapies for AIDS, only a few years after the elucidation of the life cycle of the HIV virus, is testament to the power of structure-based design.

The primary drawback with SBDD is that it is limited to drug targets that are readily crystallizable, e.g., enzymes. However, the majority of drugs target proteins are not enzymes, but rather are, e.g., integral membrane proteins, i.e., proteins that span the lipid membrane of the cell, and are difficult to manipulate outside of that lipid environment. The two largest gene familes among these integral membrane protein drug targets are G-protein coupled receptors (GPCR's) and ion channels. Beta-blockers, such as, propanolol, which are used for treating heart conditions, are the most well-known class of drugs among those targeting CPCR's; benzodiazepines, such as, diazepam, used for treating psychiatric disorders, are the most well-known class of drugs targeting ion channels.

Fundamentally, the two pieces of information that are critical for the process of SBDD is the conformation of the molecule bound to the drug target, and the types of interactions the molecule makes with the drug target. X-ray crystallography is not the sole biophysical method capable of providing such information. Macromolecular NMR has long shown promise for providing the information required for SBDD. But, macromolecular NMR has not yet fulfilled its potential despite an enormous evolution in the methodology in the past dozen years.

Purely computational approaches (“molecular modeling”) have also been used, in an attempt to infer this information critical to SBDD in the absence of direct macromolecular information. Here, too, molecular modeling has not fulfilled its potential, despite progress in the computational methodology.

Organic chemists have long relied on plastic models to enhance their understanding of the three-dimensional (“3D”) properties of molecules. The first step forward computationally beyond this was in the early 1970's, with Norman Allinger's “conformational analysis,” in which the computer would calculate the allowed 3D conformations of a molecule, based on a very-carefully tailored set of “force-field parameters” [Allinger, N. L., “Conformational Analysis. 130. MM2. A Hydrocarbon Force Field Utilizing V1 and V2 Torsional Terms”, J. Am. Chem. Soc. 1977, 99, 8127; Burket, U., Allinger, N. L., “Molecular Mechanics,” American Chemical Society Meeting: Washington, D.C., 1982]. In the late 1970's, Garland Marshall exploited conformational analysis for the purposes of drug design with the development of his “active analog approach” [Marshall, G. R., Barry, C. D., Bosshard, H. E., Dammkoehler, R. A. and Dunn, D. A., “The Conformational Parameter in Drug Design: The Active Analog Approach. in Computer-Assisted Drug Design,” ACS Symposium Series 112, eds., Olson, E. C. and Christoffersen, R. E., ACS, Washington, D.C. (1979), pp. 205-226]. In this approach, conformational analysis is performed on a set of molecules that are all active against one receptor, and interactive computer graphics was used to look for a conformation of each molecule that was suggestive of a common mode of binding to that receptor.

In the mid-1980's, this approach was extended by distilling the information present in the oriented conformations of active molecules into a “pharmacophore,” an abstract description of the 3D properties of a class of molecules that confer activity against a particular receptor [Van Drie, J. H., Weininger, D., Martin, Y. C., “ALADDIN: an integrated tool for computer-assisted molecular design and pharmacophore recognition from geometric, steric, and substructure searching of three-dimensional molecular structures,” J. Comput. Aided Mol. Des., 1989 September, 3(3):225-51]. Specially-built software called ALADDIN used this pharmacophore to search a database of existing molecules, to identify molecules which may also be active against that receptor. The use of ALADDIN to discover a novel D1 agonist in 1987 was the first example of a successful application of this method, sometimes termed ‘virtual screening’ [Martin, Y. C., “3D Database Searching in Drug Design,” J. Med. Chem., 35(12):2145-2154(1992)]. The success of ALADDIN stimulated the development of many comparable techniques, generically called “3D database searching.”

In the late 1980's and early 1990's, a number of efforts were initiated nearly-simultaneously to deal with the problem highlighted by the use of 3D database searching techniques: How can one automatically create a pharmacophore? [J. H. Van Drie, “3D Databases in Drug Discovery,” Netsci: Computers in Chemistry, 1(3) (September, 1995 issue of this electronic journal http://www.awod.com/netsci); J. H. Van Drie, “3D Databases on the Desk of the Medicinal Chemist,” Software Entwicklung in der Chemie-10, J. Gasteiger (ed.), Frankfurt: Springer-Verlag, 1996].

Marshall and co-workers described the first semi-automated procedure for determining a pharmacophore [Mayer, D., Naylor, C. B., Motoc, I., Marshall, G. R., “A unique geometry of the active site of angiotensin-converting enzyme consistent with structure-activity studies,” J. Comput. Aided Mol. Des. 1987 April; 1(1):3-16]. A group at Searle described in 1989 a semi-automated pharmacophore generation procedure called APOLLO. Another such a method called DISCO was commercialized by Tripos [Martin, Y. C., Bures, M. G., Danaher, E. A., DeLazzer, J., Lico, I., Pavlik, P. A., “A fast new approach to pharmacophore mapping and its application to dopaminergic and benzodiazepine agonist,” J. Comput. Aided. Mol. Des. 1993 February; 7(1):83-102.]. Another product Apex-3D was introduced by Biosym [Golender, V. E., Vorpagel, E. R., “3D QSAR in Drug Design: Theory, Methods and Applications,” Kubinyi, H., (Ed.), ESCOM, Leiden, 1993, pg. 137-149]. None of these efforts were noteworthy for their technical or commercial success.

However, out of the lessons learned from these initial efforts emerged a few technical successes. YAK and PRGEN grew out of the initial APOLLO efforts, and has been adopted by a number of research groups [Vedani, A., Zbinden, P., Snyder, J. P., “Pseudoreceptor modeling: A New Concept for the Three-dimensional Construction of Receptor Binding Sites,” J. Receptor Res., 1993, 13, 163-177; Snyder, J. P., Rao, S. N., Koehler, K. F., Vedani, A., “Minireceptors and Pseudoreceptors In 3D QSAR in Drug Design Theory, Methods and Applications,” Kubinyi, H., Ed.; Escom: Leiden, 1993, pp. 336-354].

DANTE went back to the original roots of Marshall's active-analog approach, and extended the work of Mayer et al, incorporating some features of the Hypothesis Generation approach [See, e.g., J. H. Van Drie, “An inequality for 3D database searching and its use in evaluating the treatment of conformational flexibility,” J. Comp.-Aided Mol. Design, 10, p. 623 (1996); J. H. Van Drie, “Strategies for the determination of pharmacophoric 3D database queries”, J. Comp.-Aided Mol. Design, 11, p. 39 (1997); J. H. Van Drie “‘Shrink-wrap’ surfaces: A new method for incorporating shape into pharmacophoric 3D database searching”, J. Chem. Inf. and Comp. Sci., 37, p. 38 (1997); J. H. Van Drie and R. A. Nugent, “Addressing the challenges of combinatorial chemistry: 3D databases, pharmacophore recognition and beyond,” SAR and QSAR in Env. Res, 9, p. 1-21 (1998)].

The primary weaknesses pharmacophore discovery methods is that they rely on two fundamental assumptions:
that the actual conformation of the molecule as it binds to the receptor is among those conformations explored in the computational conformational analysis; and
that all molecules are binding to the receptor in a common way.

Assumption 1 is generally not too problematic with modern conformational analysis techniques, but difficulties are introduced in that many conformations must be explored to ensure that this assumption is true.

Assumption 2 is a source of uncertainty in such pharmacophore methods; in principle, the methodology of DANTE is capable of detecting when this assumption is not true for a small number of molecules in the dataset, but real data is frequently noisier than the level at which the DANTE algorithm is robust.

### SUMMARY OF THE INVENTION

The present invention relates to a process for producing an optimized pharmacophore, said process comprising the steps of:
(a) selecting a first dataset comprising:
   i. chemical structure information of a plurality of compounds; and
   ii. a first quantified property of each of said plurality of compounds, wherein said first quantified property is related to the affinity of each of said plurality of compounds to a target protein;
(b) applying a first computational means to said first dataset to generate a first pharmacophore;
(c) applying a second computational means to a second dataset to produce said optimized pharmacophore, wherein said second dataset comprises:
   i. one, two or all of said first pharmacophore, said first data set and said first quantified property; and
   ii. a second quantified property for each of said plurality of compounds, wherein said second quantified property is related to the conformation of each of said plurality of compounds when it is bound to said target protein; and
(d) outputting said optimized pharmacophore to a suitable output device.

The present invention also provides processes for identifying compounds having an affinity to a target protein. The present invention also provides to processes for designing a ligand for a target protein using the optimized pharmacophore of the present invention. The present invention also provides a computer for use in desgining a ligand for a target protein using the optimized pharmacophore of the present invention.

### DETAILED DESCRIPTION OF THE INVENTION

The present invention provides a process for producing an optimized pharmacophore, said process comprising the steps of:
(a) selecting a first dataset comprising:
   i. chemical structure information of a plurality of compounds; and
   ii. a first quantified property of each of said plurality of compounds, wherein said first quantified property is related to the affinity of each of said plurality of compounds to a target protein;
(b) applying a first computational means to said first dataset to generate a first pharmacophore;
(c) applying a second computational means to a second dataset to produce said optimized pharmacophore, wherein said second dataset comprises:
   i. one, two or all of said first pharmacophore, said first data set and said first quantified property; and
   ii. a second quantified property for each of said plurality of compounds, wherein said second quantified property is related to the conformation of each of said plurality of compounds when it is bound to said target protein; and
(d) outputting said optimized pharmacophore to a suitable output device.

The optimized pharmacophore of the present invention comprises a plurality of structural constraints, wherein such constraints are useful in designing one or more ligands for a target protein.

The structural constraints of an optimized pharmacophore may be spatial constraints or interactive constraints. The term “interactive constraints” as used herein means constraints on the interaction between a compound and a target protein. Examples of “interactive constraints” include constraints relating to hydrogen bond donors/acceptors, polar/non-polar interactions or hydrophobic/hydrophilic interactions.

The term “spatial constraints” as used herein means constraints on e.g., bond angles, bond distances, inter-atom distances, molecular or conformational shape, or molecular volume.

Suitable spatial or interactive constraints of the present invention include, but are not limited to, one or more of the following:
hydrogen bond donor/acceptor interactions;
conformational shape constraints;
bond distances;
inter-atom distances;
molecular volume constraints;
hydrophobic or hydrophilic interactions;
distance constraints between atoms on the binding site of a target protein and one or more of atoms of an inhibitor;
stacking interactions between aromatic rings on an inhibitor and aromatic rings on a binding site backbone of a target protein;
orientational constraints on one or more inter-atom vectors in a molecule with respect to an external reference frame;
torsional angle constraints; or
interactions between charged atoms.

As used in the present invention, the term “target protein” means a protein suitable for the processes of the present invention. Suitable target proteins used in the processes of the present invention include, e.g., integral membrane proteins and membrane-tethered proteins. More preferably, the target proteins of the present invention are integral membrane proteins. Even more preferably, the target proteins of the present invention are GPCR's, ion channel proteins, transporters or cytokine receptors. According to another more preferred embodiment, the target proteins of the present invention are GPCR's and ion channel proteins. According to another more preferred embodiment, the target proteins of the present invention are GPCR's. According to another embodiment, the target proteins also include proteins that are not easily crystallized.

The term “ligand” as used in the present invention means a compound that has a significant affinity to a target protein. Ligands of a given target protein may be agonists, antagonists, inverse agonists, etc., of that target protein.

The processes of the present invention employ a first dataset comprising:
(i) chemical structure information of each of a plurality of compounds; and
(ii) a first quantified property related to the affinity of each of said plurality of compounds to a given target protein.

The chemical structure information present in the first dataset means information that uniquely defines the structure of each of the plurality of compounds. Such information includes, e.g., atom connectivity within each compound, inter-atom distances within each compound, etc. The chemical structure information may be pictorial, such as an output from a chemical structure drawing program. Alternatively, the chemical structure information may be numerical, alpha-numerical or any other suitable non-pictorial format that describes the structure of a compound.

According to a preferred embodiment, the first dataset comprises chemical structure information of at least 3 compounds. According to a more preferred embodiment, the first dataset comprises chemical structure information of at least 50 compounds. According to another more preferred embodiment, the first dataset comprises chemical structure information on at least 500 compounds. According to another more preferred embodiment, the first dataset comprises chemical structure information of at least 1000 compounds.

The first quantified property of a given compound is a quantified value of at least one indicator of the affinity of each compound to a given target protein. Indicators of affinity suitable for the present invention include binding constants, IC50, EC50, ligand exchange rate constants (k_{off} and kₒₙ, wherein k_{off}/kₒₙ is k_{D}, the dissociation constant), thermodynamic parameters. According to one embodiment, the first quantified property of a given compound is a quantified value of one, two or three indicators of the affinity of each compound to a given target protein. More preferably, one or two indicators of the affinity are quantified. Even more preferably, the first quantified property of a given compound is a quantified value of one indicator of the affinity of each compound to a given target protein.

According to another embodiment, the first quantified property of each of the plurality of compounds may be a quantified value of the same indicator of the affinity to a given target protein (for example, the binding constant). Or, the first quantified property of each of the plurality of compounds may be a quantified value of more than one indicator of the affinity to a given target protein. Thus, some of all compounds within a first dataset may have a first quantified property that is a quantified value of a first indicator of affinity, such as the binding constant, while the rest of all compounds within that first dataset may have a first quantified property that is a quantified value of a second indicator of affinity, such as the IC50.

Preferably, the first quantified property is selected from binding constants and IC50. Most preferably, the first quantified property is a quantified value of the binding constant of each of the plurality of compounds to a given target protein.

The first computational means according to the present invention is, typically, a molecular modeling method capable of analyzing the first dataset to produce a first pharmacophore. Molecular modeling methods useful in such analysis are known in the art. An example of such a method is DANTE. See, e.g., J. H. Van Drie, “An inequality for 3D database searching and its use in evaluating the treatment of conformational flexibility,” J. Comp.-Aided Mol. Design, 10, p. 623 (1996); J. H. Van Drie, “Strategies for the determination of pharmacophoric 3D database queries”, J. Comp.-Aided Mol. Design, 11, p. 39 (1997); J. H. Van Drie “‘Shrink-wrap’ surfaces: A new method for incorporating shape into pharmacophoric 3D database searching”, J. Chem. Inf. and Comp. Sci., 37, p. 38 (1997); J. H. Van Drie and R. A. Nugent, “Addressing the challenges of combinatorial chemistry: 3D databases, pharmacophore recognition and beyond,” SAR and QSAR in Env. Res, 9, p. 1-21 (1998). The above disclosures related to DANTE are incorporated herein by reference.

The first pharmacophore produced by the first computational means comprises a plurality of structural constraints. These are spatial or interactive constraints or both. The terms “spatial constraints” and “interactive constraints” are as defined above. But, the structural constraints of the first pharmacophore are less accurate and less precise than the structural constraints of the optimized pharmacophore. Thus, a compound that satisfied the structural constraints of the optimized pharmacophore will likely exhibit greater affinity to a target protein than a compound that only satisfies the structural constraints of the first pharmacophore.

The second dataset according to the present invention comprises:
(i) one, two or all of said first pharmacophore, said first data set and said first quantified property; and
(ii) a second quantified property for each of said plurality of compounds, wherein said second quantified property is related to the conformation of each of said plurality of compounds when bound to said target protein.

The second quantified property in the second dataset of the present invention is a quantitative value of one or more indicators directly related to one or more conformations of a compound when bound to a target protein. Examples of such indicators in the second quantified property include constraints such as inter-atom distances, torsion angles, orientation of inter-atom vectors, or suitable descriptors of conformations incorporating such constraints.

According to a preferred embodiment, the second dataset comprises:
i. one or both of said first pharmacophore and said first data set; and
ii. a second quantified property for each of said plurality of compounds, wherein said second quantified property is related to the conformation of each of said plurality of compounds when it is bound to said target protein.

According to a more preferred embodiment, the second dataset comprises:
i. said first pharmacophore; and
ii. a second quantified property for each of said plurality of compounds, wherein said second quantified property is related to the conformation of each of said plurality of compounds when it is bound to said target protein.

The selection of indicators will be guided by the particular technique used to study a compound bound to a target protein. Such techniques include solution state Nuclear Magnetic Resonance (“NMR”), Electron Paramagnetic Resonance or fluorescence spectroscopy and solid state NMR. As an illustration, the use of solution state NMR techniques for studying the bound state of a compound is described below. One of skill in the art would be well aware of other techniques that accomplish the same goal.

Different solution NMR techniques utilizing transferred Nuclear Overhauser Effect, paramagnetic probes, transferred cross-correlation, transferred residual dipolar couplings and relaxation anisotropy may be used in the present invention.

Nuclear Overhauser Effect SpectroscopY (“NOESY”)

NOESY exploits distance constraints that are a direct consequence of the particular molecular structure (i.e., different structures will lead to different sets of short inter-proton distances). Provided one has enough of these distance constraints, well-established algorithms can determine structures that are consistent with the observed inter-proton distances (see, e.g., Wuthrich, K., NMR of Proteins and Nucleic Acids (1986)). This ability to provide intra-molecular distance constraints renders NOESY a useful method for determining solution structures via NMR.

One type of NOESY technique, known as the transferred NOE method (“tNOE method”), involves performing NOESY on a binder molecule (“binder”) that binds to a target molecule. For the tNOE method to succeed, the binder/target system should satisfy the following criteria:
1. the binder is in fast chemical exchange between the free and bound states;
2. the binder is in molar excess of the target (typically ≧10:1); and
3. the target molecule has a molecular weight much larger than the binder.

If the foregoing criteria are satisfied, then a NOESY performed on the exchanging binder provides distance restraints that reflect primarily the receptor-bound conformation. These distance restraints may then submitted to standard structure-determination algorithms to yield the receptor-bound conformation. The tNOE method is attractive because chemical exchange effectively “transfers” the structural information from the bound state to the free binder NMR resonances; we need not observe the target NMR resonances at all. The free binder NMR resonances are sharp and easily detected on account of their low molecular weight. In contrast, the much higher molecular weights of the receptor and receptor-bound binder lead to typically undetectable NMR resonances. Thus, tNOE enables us to get the structures of binders bound to target proteins that are often too large for direct NMR structure determinations. See, e.g., Balram, P., Bothner-By, A. A., and Dadok, J. Am Chem. Soc. 94 4015 (1972); Balram, P., Bothner-By, A. A., and Breslow, D., J. Am. Chem. Soc. 94 4017 (1972); Campbell, A. P. and Sykes, B. D., J. Magn. Reson. 93 77-92 (1991); Clore, G. M., and Gronenborn, A. M., J. Magn. Reson. 48 402 (1983); Ni, F., Recent Developments in Transferred NOE Methods, Prog NMR Spect, 26 517-606 (1994).

Procedure for the Application of tNOE to Integral Membrane Protein Targets

The above tNOE method can be applied to the case in which the target molecule is an integral membrane protein, e.g., a GPCR or ion-channel. The plausibility of this approach for GPCR targets has been demonstrated recently in the literature (see, e.g., Kisselev, O. G et al, Light-activated rhodopsin induces structural binding motif in G protein a subunit, Proc. Natl. Acad. Sci. USA 95 4270-4275 (1998); Inooka, H. et al, Conformation of a peptide binder bound to its G-protein coupled receptor, Nat. Struct. Biol. 8 161-165 (2001)).

For integral membrane protein targets, preferably the protein can be reconstituted in a suitable membrane mimetic environment (e.g., detergent micelle or bicelle) that does not compromise the protein's natural binding properties. The combined protein/membrane-mimic system will thus satisfy criterion 3 above. It is further preferred that the binder has an aqueous solubility and K_{D} such that criteria 1 and 2 are also satisfied. Finally, the binder should preferably bind directly to the membrane protein, and not the molecules that comprise the membrane mimetic environment (e.g. lipid and/or detergent molecules).

The following steps may then be used to determine the conformation of the binder while bound to the membrane protein.

1. Preliminary experiments to assign the binder resonances. One records preliminary standard experiments on the binder/receptor sample to assign the binder proton resonances. Here, “assignment” simply refers to the process in which we identify which binder proton leads to which binder NMR resonance (signal).

2. NOESY experiment of binder/target sample. One measures a NOESY for the exchanging binder resonances. The binder/target system is assumed to obey all of the above criteria. The NOESY distance restraints obtained report primarily on the bound binder conformation. To ascertain whether any contributions from the free binder must be considered, reference experiments should be performed (step 3).

3. Reference NOESY experiments. One measures a NOESY for the binder under conditions in which the binder is not bound to the membrane protein. The purpose of this experiment is to check for potential free state contributions, which, if significant, should be subtracted from the NOESY in step 2. Two such reference experiments may be performed:
i) If there is a known tighter binder of the membrane protein (e.g. natural binder or analog thereof), then small amounts of this tighter binder can be added to the previous binder/target sample. The tighter binder will eventually displace the test binder if the two molecules for the same binding site on the membrane protein. Measurement of a NOESY then reveals just the free binder contribution to the NOESY in step 2, and also confirms that the binder binds specifically.
ii) A known tighter binder may not be available. Or, the binder binds to the target but not at the same place as the known binder. In this case, one must prepare a second NMR control sample that consists as nearly as possible of the same concentration binder as in the binder/receptor sample under identical buffer conditions. Again, one records a NOESY and the resulting NOE cross peaks yield just the free state contributions.

4. Supplementary Experiments: The NOESY experiment represents the most important and straightforward source of structural information. However, it may be complemented by other NMR experiments that also take advantage of the exchange-transferred effect. Examples include the use of paramagnetic probes, cross-correlated relaxation experiments, scalar and residual dipolar coupling experiments, relaxation anisotropy experiments etc.

5. Structure Calculations: The free binder contribution to the NOESY-based distance restraints in part 3 are subtracted out, resulting in a NOESY spectrum that contains information purely on the bound conformation. The distance restraints are input to a standard NMR structure-determination algorithm. Examples of such algorithms include the combined distance-geometry/simulated annealing methods available from commercial vendors (see e.g. Brunger, 1993). The results are an ensemble of conformations that are consistent with the tNOE data.

Paramagnetic Probes

Structural constraints related to the bound conformation may be obtained through the use of paramagnetic labels (chemical moieties with an unpaired electron). Such labels are commonly observed in EPR. In NMR, the effects of these labels are observed indirectly by their effects on spatially close NMR active nuclei. In particular, the strong magnetic moment of the unpaired electron induces broadening and/or chemical shift changes to NMR nuclei that are close in space. For example, protons within 15-20 Å of a nitroxide spin label will show broadening; these distances are, typically; over an order of magnitude from those observed by the aforementioned tNOE measurements. Quantification of this paramagnetic broadening and/or chemical shift perturbation results in distance and/or orientational constraints for the molecule containing the affected nuclei. Accordingly, one can attach the labels to either the target protein and/or binding molecule. One can compare the NMR spectra (e.g. ¹H, ¹³C, ¹⁵N, ¹⁹F) of a bound molecule in the presence and absence of a paramagnetically-labeled target. Identifying the resonances perturbed by the labeled target, and quantifying such perturbation provides structural constraints for the bound state conformation of the molecule. Alternatively, a known tightly-binding ligand to the target protein is labeled. This labeled ligand-target protein complex is screened against secondary molecules.

For a description of this technique, in general, see, Spin Label Enhanced NMR Screening, Jahnke, W., Rüdisser, S., and Zurini, M., J. Am. Chem. Soc. 123, 3149-3150 (2001); Battiste, J. L. and Wagner, G., Utilization of Site-Directed Spin Labeling and High-Resolution Heteronuclear Nuclear Magnetic Resonance for Global Fold Determination of Large Proteins with Limited Nuclear Overhauser Effect Data, Biochemistry 39, 5355-5365 (2000); and Second-Site NMR Screening with a Spin-Labeled First Ligand, Jahnke, W., Perez, L. B., Paris, C. G., Strauss, A., Fendrich, G., and Nalin, C. M. J. Am. Chem. Soc. 122, 7394-7395 (2000).

Transferred Cross-Correlation

NMR experiments that measure cross-correlated relaxation rate constants can provide torsion angle constraints. “Cross-correlation” refers to the correlation between two NMR relaxation mechanisms within the molecule. Each mechanism is defined by a vector, and the cross-correlated relaxation rate constants provide information about the torsion angle between the two vectors. An example is the cross-correlation between two vicinal ¹³C-¹H dipole-dipole interactions; the cross correlation rate constant then provides constraints on the CH—CH torsion angle. Similar to the tNOE, one can measure these rate constants for a ligand in fast exchange between the free and bound states. The exchange-averaged cross-correlated relaxation rate constants then contain information about torsion angles in the bound-state conformation. In principle, cross-correlation between any two well-known relaxation mechanisms (e.g. dipole-dipole, chemical shift anisotropy) may be exploited for gaining bound conformation angular constraints. The limiting factors are sensitivity because one must often deal with nuclei other than ¹H (e.g. ¹⁹F, ¹³C, ¹⁵N, ²H). An example in the literature that uses a uniformly ¹³C isotope-enriched ligand is found in “Application to the Determination of Sugar Pucker in an aminacylated tRNA-Mimetic Weakly Bound to EF-Tu, Carlomagno,” T., Felli, I. C., Czech, M., Fischer, R., Sprinzl, M., and Griesinger, C., J. Am. Chem. Soc. 121, 1945-1948 (1999).

Transferred Residual Dipolar Couplings

Normally, direct dipolar couplings between NMR nuclei are averaged out in liquid-state NMR samples. However, it has been shown recently that ligand-target systems may be studied in solution media in which a weak alignment is enforced on the molecules. The weak alignment reintroduces residual dipolar couplings, which then appear as additional splittings in the NMR spectra. The splittings from the residual dipolar couplings are distinct from those stemming from the well-known scalar couplings. Quantification of these residual dipolar couplings provides information about the orientation of bond vectors relative to the alignment axis of the sample. These couplings can be meausred for a molecule in rapid exchange between the free and bound states. The observed residual dipolar couplings then provide information about the orientation of ligand bond vectors within the bound state. An example in the prior art that looks at the bound conformation of an ¹⁵N-enriched peptide ligand binding to a GPCR (rhodopsin) solubilized in rod outer segment membranes is found in Koenig, B. W., Mitchell, D. C., Konig, S., Grzesiek, S., Litman, B. J., and Bax, A., “Measurement of dipolar couplings in a transducin peptide fragment weakly bound to photoactivated rhodopsin,” J. Biolmol. NMR. 16(2), 121-125 (2000).

Relaxation Anisotropy

NMR relaxation rate constants (e.g. of ¹⁵N, ¹³C, ¹⁹F, ¹H) have a dependence on overall molecular shape. This effect is more pronounced for larger molecules that possess a significant shape anisotropy (anisotropy, as used herein, means that the molecule cannot be modeled as a sphere). Molecules that exchange on and off a large target protein transiently experience the overall shape anisotropy of the target. Accordingly, the relaxation rate constants of a molecule in the presence and absence of target can be compared. The observed perturbation of those rate constants due to the shape anisotropy of the target can then be quantified. That perturbation provides information about the orientations of the molecule bond vectors relative to the diffusion tensor principle axes of the target molecule. Thus, orientational constraints for the bound ligand conformation can be obtained. See, Tjandra, N., Garrett, D. S., Gronenborn, A. M., Bax, A. and Clore, G. M., “Defining long range order on NMR structure determination from the dependence of heteronuclear relaxation times on rotational diffusion anistropy,” Nat Struct Biol 4(6), 443 449 (1997).

Another technique that may be used to obtain information about the bound conformation of a compound is solid state NMR. Recent advances allow the determination of interatomic distances and torsion angles for bound ligands that are isotopically enriched. See, e.g., Grobner, G., Burnett, I. J., Glaubitz, C., Choi, G., Mason, A. J., Watts A., “Observations of light-induced structural changes of retinal within rhodopsin”, Nature 405(6788), 810-813 (2000); Balbach, J. J., Ishii, Y., Antzukin, O. N., Leapman, R. D., Rizzo, N. W., Dyda F., Reed, J. and Tycko, R., “Amyloid Fibril Formation by Ab16-22, a Seven-Residue Fragment of the Alzheimer's b-Amyloid Peptide, and Structural Characterization by Solid State NMR”, Biochemistry 39, 13748-13759 (2000); and Weliky, D. P., Bennet, A. E., Zvi, A., Anglister, J., Steinbach, P. J., Tycko, R., “Solid-state NMR evidence for an antibody-dependent conformation of the V3 loop of HIV-1 gp120” Nat Struct Biol 6, 141-145 (1999).

The second computational means is key to the advantages conferred by the processes of the present invention. It typically comprises suitable molecular modeling software. The software is capable of analyzing the second quantified property, together with one, two or all of said first pharmacophore, said first quantified property, and said chemical structure information.

Application of the second computational means produces the optimized pharmacophore for a target protein. As noted above, the second quantified property relates to the affinity of each of the plurality of molecules with a target ligand. Thus, the use of the second quantified property in the application of the second computational means has the effect of refining the structural constraints in the first pharmacophore. For this reason, the structural constraints of the optimized pharmacophore are more accurate and more precise when compared to the structural constraints in the first pharmacophore. The enhanced accuracy and precision are key advantages in studying proteins such as integral membrane proteins and membrane-tethered proteins.

According to an alternate embodiment, the present invention provides a process for producing an optimized pharmacophore, said process comprising the steps of:
(A) applying a second computational means to a third dataset, wherein said third dataset comprises:
   (a) at least one of:
      i. chemical structure information of a plurality of compounds;
      ii. a first quantified property of each of said plurality of compounds, wherein said first quantified property is related to the affinity of each of said plurality of compounds to a target protein; and
      iii. a first pharmacophore; and
   (b) a second quantified property for each of said plurality of compounds, wherein said second quantified property is related to the conformation of each of said plurality of compounds when it is bound to said target protein; and
(B) outputting said optimized pharmacophore to a suitable output device.

According to a preferred embodiment, said third dataset comprises:
(a) a first pharmacophore; and
(b) a second quantified property for each of said plurality of compounds, wherein said second quantified property is related to the conformation of each of said plurality of compounds when it is bound to said target protein.

According to a more preferred embodiment, said third dataset comprises:
(a) chemical structure information of a plurality of compounds; and
   a first quantified property of each of said plurality of compounds, wherein said first quantified property is related to the affinity of each of said plurality of compounds to a target protein; and
(b) a second quantified property for each of said plurality of compounds, wherein said second quantified property is related to the conformation of each of said plurality of compounds when it is bound to said target protein.

The structural constraints present in the optimized pharmacophore of the present invention are useful in designing one or more ligands for a given target protein. There are a plurality of approaches known in the art that may be applied to the structural constraints of an optimized pharmacophore of the present invention to design one or more ligands for a target protein. Three such approaches are illustrated in Scheme 1 and described in detail below.

Approach 1

According to one embodiment, the optimized pharmacophore of a target protein may be employed by one of skill in the art in manually designing a compound having an affinity to a target protein.

Approach 2

According to an alternate embodiment, the present invention provides a process for identifying a compound having an affinity to a target protein, said method comprising the steps of:
(step 1) selecting an optimized pharmacophore for said target protein;
(step 2), virtually screening each of a plurality of molecular structures in a database against said optimized pharmacophore to identify a suitable molecular structure;
(step 3) outputting said suitable molecular structure to a suitable output device. These three steps are discussed below in detail.

Step 1

The processes described hereinabove provide an optimized pharmacophore for a given target protein. Thus, depending on the target protein selected for ligand design, the corresponding optimized pharmacophore according to the present invention is selected.

Step 2

Virtual screening, as used herein, means a 3D database search of a plurality of molecular structures to identify one or more suitable molecule structures. A suitable molecular structure is one that substantially satisfies the structural constraints of an optimized pharmacophore. The 3D database typically comprises molecular structures of compounds that are commercially available or are known in the prior art literature. The compounds in the 3D database may have been selected at random or may have been selected based on one or more criteria, such as compounds with known affinity for the target protein. Commercially available modeling software, such as ALADDIN, ISIS/CFS, Catalyst or CHEM-3 DBS, may be readily used to perform the virtual screening. Typically, the structural constraints of the optimized pharmacophore are input into the modeling software. The modeling software virtually screens each molecular structure in the 3D database.

Step 3

One or more suitable molecular structure so identified in the virtual screening step is the outputted to an appropriate output device such as a printer or a CRT display device.

Approach 3

According to an alternate embodiment, the present invention provides a de novo approach to designing a ligand for a target protein. Thus, the present invention provides a process for identifying a compound structure having an affinity to a target protein, said method comprising the steps of:
(step 1) selecting an optimized pharmacophore for said target protein;
(step 2) identifying a discrete structure element corresponding to each structural constraint of said optimized pharmacophore and creating therewith a molecular scaffold;
(step 3) mining said molecular scaffold to identify a suitable molecular structure;
(step 4) outputting said molecular structure to a suitable output device.

Step 1

The processes described hereinabove provide an optimized pharmacophore for a given target protein. Thus, depending on the target protein selected for ligand design, the corresponding optimized pharmacophore according to the present invention is selected.

Step 2

The structural constraints of the optimized pharmacophore are transformed into a plurality of molecular scaffolds. This step involves two sub-steps, as described below.

(i) The structural constraints of the optimized pharmacophore, either individually or in combination, may be readily correlated to a corresponding discrete structural element that satisfy the structural constraints. A discrete structural element, as used herein, is an atom or a group of atoms having a defined connectivity. This correlation may be done manually or using suitable computational means known in the art. For example, hydrogen bond donor/acceptor interactions may be translated into suitable atom(s) that provide such interactions, e.g., —OH. Or, conformational constraints may, e.g., preclude a 4- or 5-membered ring in favor of a 6-membered ring. Or, distance constraints may favor a C1-C6 alkyl chain, while disfavoring longer alkyl chains. Or, torsion angle constraints may favor certain substituents over others. Thus, in one embodiment, each structural constraint in the optimized pharmacophore will correspond to one or more discrete structural elements. Or, in some instances, two or more structural constraints in the optimized pharmacophore may, in combination, correspond to one or more discrete structural elements.

One of skill in the art will be well aware that a plurality of discrete structural elements may each satisfy the same structural constraint(s). For example, a hydrogen bond donor constraint may be satisfied by, e.g., three different structural elements, namely, —OH, —SH or —NH—. Or, an aromatic stacking interaction constraint may be satisfied by structural elements such as phenyl, pyridyl, furyl, or any other heteroaromatic ring. A hydrophobic interaction constraint coupled with a molecular volume constraint may favor a structural element such as secondary or tertiary lower alkyl. A hydrophilic interaction constraint may favor hydroxy or amino substitution at an appropriate structural element in a molecule.

(ii) A combination of a set of structural elements that, together, satisfies substantially the structural constraints in the optimized pharmacophore, produces a molecular scaffold; i.e., a structural backbone that represents a de minimus set of structural features necessary in a compound for substantially satisfying the structural constraints of the optimized pharmacophore. The various combinations of structural elements that satisfy the structural constraints in the optimized pharmacophore provide a plurality of molecular scaffolds. For example, for a given target protein, the corresponding optimized pharmacophore may contain two structural constraints, namely, a six-membered saturated ring and hydrogen bond donor attached thereto. The six-membered saturated structural constraint can be satisfied by a cyclohexyl ring. As noted above, three different discrete structural elements can satisfy the hydrogen bond donor ring, namely, —OH, —SH and —NH—. Thus, the discrete structural elements identified in this example may be readily converted into at least 3 scaffolds; namely, cyclohexanol, cyclohexylthiol and cyclohexylamine.

Step 3

The plurality of molecular scaffolds identified in the previous step is readily mined to identify one or more suitable molecular structures. Software methods known in the art may be readily employed for such mining.

Step 4

The suitable molecular structures so identified are outputted to a suitable output device, such as a printer or a CRT-device.

When one or more suitable molecular structures are identified using the above processes, the compound with that structure is readily assayed using conventional biological techniques to determine the degree of affinity to a target protein. The key advantage to this approach is that because the compounds contain structural elements favoring affinity to the target protein, the probability of success in subsequent optimization of activity is significantly increased. Subsequent optimization is readily accomplished by conventional structure-based drug design (see, Scheme 1) to produce ligands for a target protein.

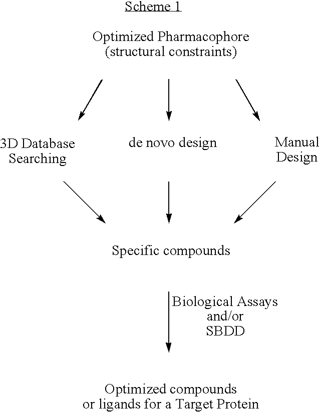

The design of ligands using the processes of the present invention is especially advantages for integral membrane proteins and membrane-tethered proteins. These proteins are not readily studied by conventional methods because they are difficult to crystallize by methods known in the prior art. Thus, design of ligands for these proteins by conventional modeling methods alone seldom provides satisfactory results. The processes of the present invention employ conventional modeling methods to produce a first pharmacophore. But, this first pharmacophore is further refined using the second quantified property to produce the optimized pharmacophore. This step is the key advantage conferred by the present invention. Thus, the optimized pharmacophore is a valuable starting point for subsequent structure based design of ligands for proteins such as the integral membrane proteins and membrane-tethered proteins.

According to another embodiment, the present invention provides a computer for designing a ligand for a target protein, said computer comprising:
(a) a machine-readable data storage medium comprising a data storage material encoded with machine-readable data, wherein said data comprises an optimized pharmacophore;
(b) a working memory for storing a computational means for processing said machine-readable data;
(c) a central-processing unit coupled to said working memory and to said machine-readable data storage medium for processing said machine-readable data; and
(d) an output device coupled to said central-processing unit for outputting the results of step (c).

Scheme 2 demonstrates one version of these embodiments. System 19 includes a computer 17 comprising a central processing unit (“CPU”) 5, a working memory 6 which may be, e.g, RAM (random-access memory) or “core” memory, mass storage memory 10 (such as one or more disk drives or CD-ROM drives or DVD's), one or more display terminals 7, one or more keyboards 9, one or more input lines 4, and one or more output lines 11, all of which are interconnected by a conventional bidirectional system bus 8.

Input hardware 16, coupled to computer 17 by input lines 4, may be implemented in a variety of ways. Machine-readable data of this invention may be inputted via the use of a modem or modems 21 connected by a telephone line or dedicated data line 1. Alternatively or additionally, the input hardware 16 may comprise CD-ROM drives or disk drives or DVD's 2. In conjunction with display terminal 7, keyboard 9 may also be used as an input device.

Output hardware 18, coupled to computer 17 by output lines 11, may similarly be implemented by conventional devices. By way of example, output hardware 18 may include a display terminal 7 for displaying the results of the processes of the present invention. Output hardware might also include a printer 20, so that hard copy output may be produced, or a disk drive 14, to store system output for later use.

In operation, CPU 5 coordinates the use of the various input and output devices 16, 18, coordinates data accesses from mass storage 10 and accesses to and from working memory 22, and determines the sequence of data processing steps. A number of programs may be used to process the machine-readable data of this invention.

According to a preferred embodiment, the computer of the present invention has a printer or a CRT-display device or an LCD device as the output device.

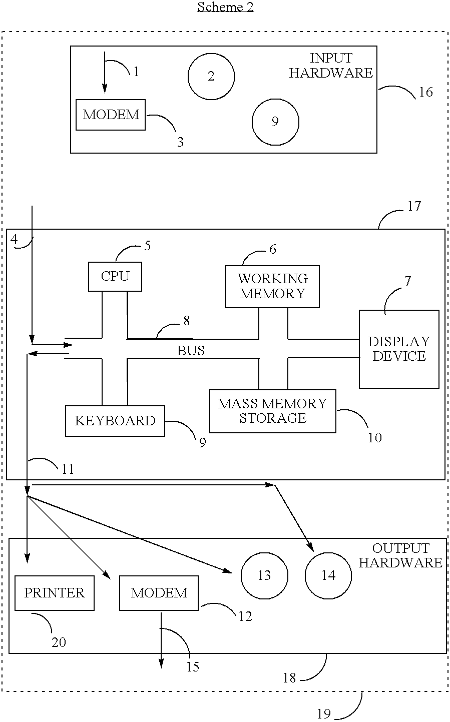

According to another embodiment, the present invention provides an iterative process for identifying optimized compounds, said process comprising the steps of:
A. associating at least one metric to each of a first plurality of compounds, wherein said metric has a first value;
B. using chemical structure information of said first plurality of compounds, a first quantified property and a second quantified property to produce an optimized pharmacophore;
C. using said optimized pharmacophore to identify a second plurality of compounds, wherein each said compound has a second value for said metric;
D. performing steps B and C recursively until said second value for said metric is an improvement over said first value;
wherein said recursion comprises the steps of:
   (i) performing step B by replacing said first chemical structure information of said first plurality of compounds with chemical structure information of said second plurality of compounds; and
   (ii) repeating step C using said optimized pharmacophore of recursive step B;
E. outputting said second plurality of compounds to a suitable output device.

The term “optimized compounds” as used herein means compounds that have demonstrable affinity to a target protein. Such affinity is demonstrated by, e.g., conventional assays known in the art.

In step A of the iterative process above, a first plurality of compounds is selected. One or more suitable metrics is/are selected for each of such compounds. The value of the metric(s) is determined for each compound. For example, for a set of 5 compounds, binding constant against a target protein and aqueous solubility may be the two metrics selected therefor. Thus, each of the 5 compounds will have an initial value (first value) for binding constant and aqueous solubility.

In step B of the iterative process above, an optimized pharmacophore for the target protein is produced using chemical structure information of said first plurality of compounds, a first quantified property and a second quantified property. This step of producing the optimized pharmacophore is as described supra. Thus, in the example above, the chemical structure of the 5 compounds, coupled with a first quantified property and a second quantified property, can be used to produce an optimized pharmacophore using the processes of the present invention.

In step C of the iterative process above, the optimized pharmacophore of step B is used to identify a second plurality of compounds. Such identification may be readily performed using, e.g., the methods of Scheme 1. Subsequently, the same metrics, as used in the first plurality of compounds in step A, are measured (second value) for each of the second plurality of compounds. Thus, in the example above, the optimized pharmacophore is used to identify a second plurality of compounds using the methods of Scheme 1. For each of this second plurality of compounds, the same metrics, i.e., binding constant and aqueous solubility in this example, are measured (second value).

In step D, the second value is compared with the first value for each metric. If the second value is deemed an improvement; i.e., if the second value of a metric for a compound identified in step C, in comparison with the first value of that metric in step A, renders that compound more desirable as a ligand, then that second value is an improvement. If the second value is not deemed an improvement, then the compounds identified in step C are used as input for step B, and the steps B and C are repeated. Thus, this iterative process is performed until a second plurality of compounds having an improved value for the metrics is identified.

In step D, the second plurality of compounds, having the second value deemed an improvement, are outputted to a suitable device. The outputted compounds compounds are optmized compounds, i.e., compounds having a demonstrable affinity to a target protein.

According to a preferred embodiment, said metric is selected from novelty, pharmacokinetic property, biological property, physical property, chemical property.

According to a more preferred embodiment said biological property is selected from binding constant, IC50, EC50 and rate constant.

According to a more preferred embodiment said physical property is selected from molecular weight, solubility, melting point, logP.

According to a more preferred embodiment said pharmacokinetic property is selected from propensity for biotransformation, membrane permeability, ability to cross blood-brain barrier and bioavalibility.

According to a preferred embodiment, said metric is novelty. “Novelty” as used herein means a structural distinction that renders a compound different from another in either atom connectivity or atom constituents or both. One of skill in the art will be aware of computational means known in the art which quantify the structural similarity/differences between two compounds. Thus, according to a preferred embodiment, the recursive process of identifying optimized compounds comprises the step of searching the prior art for compounds that negate the novelty of the identified compound. Novelty is negated if a prior art compound is identical to a compound identified by step C above (i.e., the second value is deemed not an improvement). If such a search does not negate the novelty of the identified compound, then that identified compound is considered an improvement over the compound of the previous iteration, and then is outputted to a suitable output device.

While we have hereinabove presented a number of embodiments of this invention, it is apparent that our basic construction can be readily altered to provide other embodiments that utilize the methods of this invention. Therefore, it will be appreciated that the scope of this invention is to be defined by the claims appended hereto rather the specific embodiments which been presented hereinabove.

1. A process for producing an optimized pharmacophore, said process comprising the steps of:
(a) selecting a first dataset comprising:
i. chemical structure information of a plurality of compounds; and
ii. a first quantified property of each of said plurality of compounds, wherein said first quantified property is related to the affinity of each of said plurality of compounds to a target protein;
(b) applying a first computational means to said first dataset to generate a first pharmacophore;
(c) applying a second computational means to a second dataset to produce said optimized pharmacophore, wherein said second dataset comprises:
i. one, two or all of said first pharmacophore, said first data set and said first quantified property; and
ii. a second quantified property for each of said plurality of compounds, wherein said second quantified property is related to the conformation of each of said plurality of compounds when it is bound to said target protein; and
(d) outputting said optimized pharmacophore to a suitable output device.

2. The process according to claim 1, wherein said second dataset comprises:
i. one or both of said first pharmacophore and said first data set; and
ii. a second quantified property for each of said plurality of compounds, wherein said second quantified property is related to the conformation of each of said plurality of compounds when it is bound to said target protein.

3. The process according to claim 2, wherein said second dataset comprises second dataset comprises:
i. said first pharmacophore; and
ii. a second quantified property for each of said plurality of compounds, wherein said second quantified property is related to the conformation of each of said plurality of compounds when it is bound to said target protein.

4. A process for producing an optimized pharmacophore, said process comprising the steps of:
(A) applying a second computational means to a third dataset, wherein said third dataset comprises:
(a) at least one of:
i. chemical structure information of a plurality of compounds;
ii. a first quantified property of each of said plurality of compounds, wherein said first quantified property is related to the affinity of each of said plurality of compounds to a target protein; and
iii. a first pharmacophore; and
(b) a second quantified property for each of said plurality of compounds, wherein said second quantified property is related to the conformation of each of said plurality of compounds when it is bound to said target protein; and
(B) outputting said optimized pharmacophore to a suitable output device.

5. The process according to claim 4, wherein said third dataset comprises:
(a) a first pharmacophore; and
(b) a second quantified property for each of said plurality of compounds, wherein said second quantified property is related to the conformation of each of said plurality of compounds when it is bound to said target protein.

6. The process according to claim 4, wherein said third dataset comprises:
(a) chemical structure information of a plurality of compounds; and
a first quantified property of each of said plurality of compounds, wherein said first quantified property is related to the affinity of each of said plurality of compounds to a target protein; and
(b) a second quantified property for each of said plurality of compounds, wherein said second quantified property is related to the conformation of each of said plurality of compounds when it is bound to said target protein.

7. A process for identifying a compound having an affinity to a target protein, said method comprising the steps of:
i. selecting an optimized pharmacophore for said target protein;
ii. virtually screening each of a plurality of molecular structures in a database against said optimized pharmacophore to identify a molecular structure having structural features that substantially satisfy structural constraints of said optimized pharmacophore;
iii. outputting said molecular structure to a suitable output device.

8. A process for identifying a compound structure having an affinity to a target protein, said method comprising the steps of:
i. selecting an optimized pharmacophore for said target protein;
ii. identifying a discrete structure element corresponding to each structural constraint of said optimized pharmacophore and creating therewith a molecular scaffold;
iii. mining said molecular scaffold to identify a molecular structure having structural features that substantially satisfy structural constraints of said optimized pharmacophore;
iv. outputting said molecular structure to a suitable output device.

9. A process for designing a ligand for a target protein, comprising the step of identifying a compound whose molecular structure substantially satisfies structural constraints of an optimized pharmacophore for said target protein.

10. The process according to any one of claims 1-9, wherein said target protein is an integral membrane protein or a membrane-tethered protein.

11. The process according to claim 10, wherein said target protein is an integral membrane protein.

12. The process according to claim 11, wherein said target protein is selected from GPCR or ion-channel proteins.

13. The process according to claim 10, wherein said target protein is a membrane-tethered protein.

14. The process according to claim 13, wherein said target protein is selected from transporter proteins or cytokine receptors.

15. The process according to any one of claims 1-4 or 6, wherein said first quantified property is selected from binding constant, EC50, or IC₅₀.

16. The process according to any one of claims 1-6, wherein said second quantified property is a quantitative value related to inter-atom distances, torsion angles, dihedral angles.

17. The process according to claim 16, wherein said second quantified property is a quantitative value related to inter-atomic distances.

18. A computer for designing a ligand for a target protein, said computer comprising:
(a) a machine-readable data storage medium comprising a data storage material encoded with machine-readable data, wherein said data comprises:
(i) an optimized pharmacophore; and
(ii) a plurality of molecular structures;
(b) a working memory for storing a computational means for processing said machine-readable data;
(c) a central-processing unit coupled to said working memory and to said machine-readable data storage medium for processing said machine-readable data to identify a molecular structure using said instructions; and
(d) an output device coupled to said central-processing unit for outputting the results of step (c).

19. The computer according to claim 18, wherein said plurality of molecular structures is part of a database.

20. The computer according to claim 18, wherein said output device is a printer or a CRT-display device or an LCD device.

21. A process for identifying optimized compounds, said process comprising the steps of:
A. associating at least one metric to each of a first plurality of compounds, wherein said metric has a first value;
B. using chemical structure information of said first plurality of compounds, a first quantified property and a second quantified property to produce an optimized pharmacophore;
C. using said optimized pharmacophore to identify a second plurality of compounds, wherein each said compound has a second value for said metric;
D. performing steps B and C recursively until said second value for said metric is an improvement over said first value;
wherein said recursion comprises the steps of:
(i) performing step B by replacing said first chemical structure information of said first plurality of compounds with chemical structure information of said second plurality of compounds; and
(ii) repeating step C using said optimized pharmacophore of recursive step B;
D. outputting said second plurality of compounds to a suitable output device.

22. The process according to claim 21, wherein said metric is selected from novelty, pharmacokinetic property, biological property, physical property, chemical property.

23. The process according to claim 22, wherein said biological property is selected from binding constant, IC50, EC50 and rate constant.

24. The process according to claim 22, wherein said physical property is selected from molecular weight, solubility, melting point, or logP.

25. The process according to claim 22, wherein said pharmacokinetic property is selected from propensity for biotransformation, membrane permeability, ability to cross blood-brain barrier and bioavalibility.
